(19) United States
(12) Patent Application Publication
(10) Pub. No.: US 2025/0120846 A1
(43) Pub. Date: Apr. 17, 2025

(54) FLUID COLLECTION SYSTEM INCLUDING A CLEANING SYSTEM AND METHODS

(71) Applicant: PUREWICK CORPORATION, Covington, GA (US)

(72) Inventors: Kyle Daw, Smyrna, GA (US); James David Hughett, Conyers, GA (US)

(21) Appl. No.: 18/927,083

(22) Filed: Oct. 25, 2024

Related U.S. Application Data
(63) Continuation of application No. 17/664,914, filed on May 25, 2022, now Pat. No. 12,150,885.
(60) Provisional application No. 63/193,406, filed on May 26, 2021.

(51) Int. Cl. A61F 5/441; A61F 5/44; B08B 9/032; B08B 9/08

(52) U.S. Cl. CPC A61F 5/441; A61F 5/4404; B08B 9/0321; B08B 9/08; B08B 2209/032; B08B 2209/08

(57) ABSTRACT

A fluid collection system may include a fluid collection assembly configured to receive urine from a user, a container configured to sore the urine collected from the fluid collection assembly, a collection tube disposed between the fluid collection assembly and the container, and a bi-directional pump. The fluid collection system operates in a first configuration to draw urine from the fluid collection assembly into the container and a second configuration to drain the urine and clean the fluid collection system.

### CROSS-REFERENCE TO RELATED APPLICATIONS

This application is a continuation U.S. application Ser. No. 17/664,914 filed on May 25, 2022, which claims priority to U.S. Provisional Patent Application No. 63/193,406 filed on May 26, 2021, the disclosure of which is incorporated herein, in its entirety, by this reference.

### BACKGROUND

An individual may have limited or impaired mobility such that typical urination processes are challenging or impossible. For example, the individual can have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing. In such cases, fluid collection devices are used to collect fluid from the individual.

Currently, the disposal of collected fluid and the cleaning of fluid collection devices is done manually, requiring the user or caregiver to handle the container and potentially exposing them to the contents thereof. The user or caregiver can be required to perform extensive cleaning procedures. The cleaning procedures can cause further exposure to the contents of the container.

### SUMMARY

Embodiments disclosed herein are related to assemblies, systems, and methods of using a fluid collection system for the collection and disposal of urine. In an embodiment, a fluid transfer assembly includes a fluid collection assembly configured to receive urine from a user. The fluid collection assembly includes a container configured to store urine collected from the fluid collection assembly. A collection tube may be disposed between the fluid collection assembly and the container. The fluid collection system further includes a bi-directly pump. The pump may operate in a first configuration to draw urine from the fluid collection assembly into the container and a second configuration to drain the urine and clean the fluid collection system.

In an embodiment, a system for collecting and disposing urine includes a urine collection device configured to collect urine discharged from a user. The system for collecting and disposing urine includes a container fluidly coupled to the urine collection device and configured to store urine. The container may include a fluid inlet, a fluid outlet, and a vacuum port. The system for collecting and disposing urine further includes a bi-directional pump including a first mode and a second mode, wherein the first mode is configured to draw urine from the urine collection device into the container and the second mode is configured to discharge the urine from the container and clean the system.

In an embodiment, a method for cleaning a fluid collection system includes changing the configuration of a urine collection device from a first configuration to a second configuration. The urine collection device includes a fluid collection assembly configured to receive urine from a user, a container configured to store the urine collected from the fluid collection assembly, a collection tube disposed between the fluid collection assembly and the container, a cleaning tube configured to expel the urine from the container, and a bi-directional pump. The pump may operate in a first configuration to draw urine from the fluid collection assembly into the container and a second configuration to drain the urine and clean the fluid collection system. The method for cleaning the fluid collection system also includes initiating the pump to expel urine from the container through the cleaning tube and placing a detergent into the container. The method for cleaning the fluid collection system further includes initiating the pump to cycle the detergent throughout the fluid collection system and removing the detergent from the fluid collection fluid collection system.

Features from any of the disclosed embodiments can be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a block diagram of a fluid collection system in a first configuration, according to an embodiment.

FIG. 1B is a block diagram of a fluid collection system in a second configuration, according to an embodiment.

FIG. 2 is an isometric view of a fluid collection system, according to an embodiment.

FIG. 3 is a schematic view of a control panel, according to an embodiment.

FIG. 4 is a schematic diagram of a fluid collection system, according to an embodiment.

FIG. 5 is isometric view with a cutaway showing a drain tube retraction system, according to an embodiment.

FIG. 6 is an isometric view of a fluid collection system illustrating a chamber configured to hold detergent, according to an embodiment.

FIG. 7 is a flow diagram of a method for cleaning a fluid collection system, according to an embodiment.

### DETAILED DESCRIPTION

Embodiments disclosed herein are related to assemblies, systems, and methods of using fluid collection systems. Specifically, the systems and methods described herein relate to the cleaning of fluid collection systems. Male and female urine collection devices frequently require emptying and cleaning. The disposal of urine and the cleaning of collection devices has traditionally been performed manually by a user or caregiver, which unnecessarily exposes the individual to the contaminants.

According to one or more embodiments, a fluid collection system may include a fluid collection assembly configured to receive a fluid from a user. The fluid collection assembly may be coupled to a container configured to store the urine collected from the fluid collection assembly. The fluid collected by the fluid collection assembly may include urine. The fluid(s) collected by the fluid collection assembly may also include at least one of vaginal discharge, penile discharge, reproductive fluids, blood, sweat, or other bodily fluids. The fluid collection system may be configured in a first configuration to draw urine from the fluid collection assembly and into the container.

To reduce maintenance of the fluid collection system and exposure to the fluids in the collection device, the fluid collection system may include a second configuration to drain the urine from the container and to clean the fluid collection system. The first configuration and the second configuration may include a bi-directional pump. The bi-directional pump may operate in the first configuration to draw the urine from the fluid collection assembly into the container and the second configuration to drain the urine and flush water and/or a cleaning solution through the fluid collection system. The fluid collection system may also include a drain tube coupled to the container configured to expel the content thereof and clean the container.

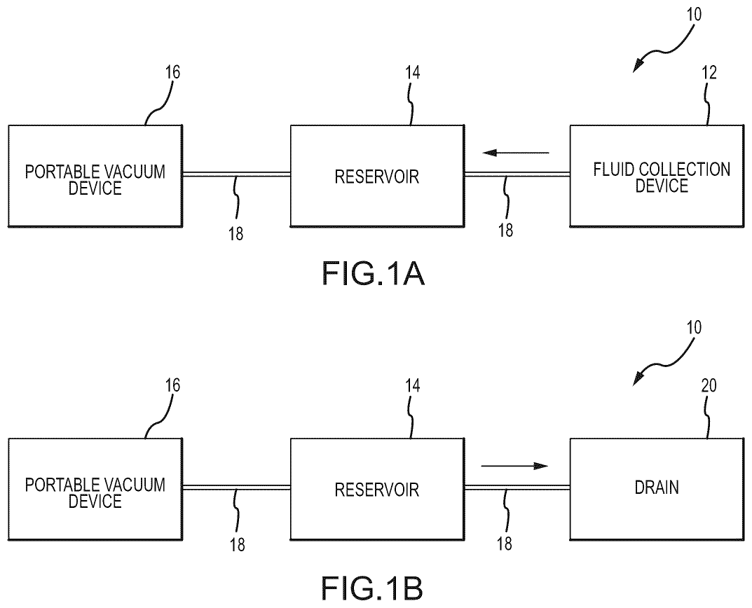

FIG. 1A is a block diagram of a fluid collection system 10 in a first configuration, according to an embodiment. The fluid collection system 10 may include a fluid (e.g., urine) collection assembly 12 (e.g., any of the fluid collection assemblies disclosed herein), a container 14 (or reservoir), and a vacuum source 16 (e.g. a pump). The fluid collection assembly 12, the fluid collection container 14, and the pump 16 may be fluidly coupled to each other via one or more collection tubes 18 disposed between the fluid collection assembly 12 and the container 14. For example, fluid collection assembly 12 may be operably coupled to one or more of the container 14 or the pump 16 via the collection tube 18. In some embodiments, the pump 16 may be coupled directly to the fluid collection container 14. Fluid (e.g., urine or other bodily fluids) collected in the fluid collection device 10 may be removed from the fluid collection device 10 via the collection tube 18 coupled to the fluid collection device 12. Suction force may be introduced into the chamber of the fluid collection device 12 via the inlet of the tube 18 responsive to suction (e.g., vacuum) force applied at the outlet of the tube 18.

The suction force may be applied to the outlet of the tube 18 by the pump 16 either directly or indirectly. The suction force may be applied indirectly via the container 14. For example, the outlet of the tube 18 may be disposed within or fluidly coupled to an interior region of the container 14 and an additional tube 18 may extend from the fluid collection container 14 to the pump 16. Accordingly, the pump 16 may apply suction to the fluid collection device 12 via the container 14. The suction force may be applied directly via the pump 16. For example, the outlet of the tube 18 may be disposed within the pump 16. An additional tube 18 may extend from the pump 16 to a point outside of the fluid collection device 12, such as to the container 14. In such examples, the pump 16 may be disposed between the fluid collection device 12 and the container 14.

The pump 16 may be in fluid communication with the interior region of the container 14 and may be configured to pull at least a partial vacuum on the interior region of the container 14 effective to draw the fluid from the fluid collection device 12 through the tube 18 into the container 14. In some embodiments, the vacuum source 16 may be coupled directly to the container 14, or the tube 18 may fluidly couple the vacuum source 16 with the interior region of the container 14.

The pump 16 may include one or more of a manual vacuum pump, and electric vacuum pump, a diaphragm pump, a centrifugal pump, a displacement pump, a magnetically driven pump, a peristaltic pump, or any pump configured to produce a vacuum. For example, the pump may include a diaphragm pump having a minimum pumping speed of 25 ml/second. In some embodiments, the pump 16 includes a variable speed pump and/or a continuous pump. For example, the pump 16 may include a variable speed pump. The pump 16 may provide a vacuum or suction to remove fluid from the fluid collection assembly 12. In some examples, the pump 16 may be powered by one or more batteries operatively coupled to the pump. In some embodiments, the battery may include a lithium ion battery. In some embodiments, the battery may be alkaline or rechargeable.

The pump 16 may provide a vacuum or suction to remove fluid from the fluid collection device 12. In some examples, the pump 16 may be powered by one or more of a power cord (e.g., connected to a power socket), one or more batteries, or even manual power (e.g., a hand operated vacuum pump). In some examples, the pump 16 may be sized and shaped to fit within a housing configured to include the container 14 and the pump 16. For example, the vacuum source 16 may include one or more miniaturized pumps or one or more micro pumps. The pump 16 disclosed herein may include one or more of a switch, a button, a plug, a remote, or any other device suitable to activate the pump 16. In some embodiments, the device to activate the pump 16 is located on the housing.

In some embodiments, the pump 16 may be coupled directly to the fluid collection container 14. The fluid collection container 14 may be sized and shaped to retain a fluid therein. The fluid collection container 14 may include a bag (e.g., drainage bag), a bottle or cup (e.g., collection jar), or any other enclosed container for storing bodily fluid(s) such as urine. In some embodiments, the fluid collection container 14 may include a generally rigid exterior housing and an interior portion configured to contain the fluid. The fluid collection container 14 may be opaque or clear according to different embodiments and may include a generally rectangular front or rear profile or be cylindrical. The fluid collection container 14 may be reusable and dishwasher safe, and may include a generally rigid material such as polycarbonate, plastic, rubber, metal, glass, combinations thereof, or any other suitable materials.

In some examples, the collection tube 18 may extend from the fluid collection device 12 and attach to the container 14 at a first point therein. The collection tube 18 may include a flexible tube. In some embodiments, at least a portion of the collection tube 18 may be substantially opaque, thereby inhibiting viewing of the fluid within the collection tube 18. An additional collection tube 18 may attach to the container 14 at a second point thereon and may extend and attach to the vacuum source 16. Accordingly, a vacuum (e.g., suction) may be drawn through fluid collection device 12 via the container 14. Fluid, such as urine, may be drained from the fluid collection device 12 using the vacuum source 16.

Referring to FIG. 1B, in some embodiments, the fluid collection system 10 may include a second configuration. In the second configuration, the fluid collection assembly 12 may be removed from the system, and disposed. In some embodiments, the fluid collection assembly 12 may be cleaned separately and restored to service. In other embodiments, the fluid collection assembly 12 may be replaced. In some embodiments, the pump 16 may be coupled directly to the fluid collection container 14. Fluid (e.g., urine or other bodily fluids) collected in the fluid collection device 10 may be removed from the container 14 via the drain tube 20 coupled to the container 14. In other embodiments, the fluid may be removed from the container 14 by opening the container 14 and pouring out the fluid directly.

The fluid collection system 10 may include a drain tube 20 that is in fluid communication with the container 14. The fluid collection system 10 in the second configuration may reverse the direction of the fluid flow with reference to the first configuration such that the fluid may be drained from the container 14 and out of the fluid collection system 10.

A positive pressure force may be applied to the container 14 by the pump 16 either directly or indirectly to assist the removal of the fluid from the container 14. The pressure force may be applied directly to the container 14. For example, the drain tube 20 may be fluidly coupled to an interior region of the container 14 and an additional collection tube 18 may extend from the fluid collection container 14 to the pump 16. Accordingly, the pump 16 may apply a positive pressure to the container 14 via the collection tube 18. The positive pressure may be applied directly via the pump 16. For example, the pump 16 may draw air from the atmosphere directly or through a collection tube 18 and discharge the air into the container 14 at a positive pressure. The positive pressure within the container 14 may cause the fluid to flow out of the drain tube 20. In this manner, the fluid collection system 10 may also be flushed with water and/or a cleaning detergent if the collection tube 18 draws fluid from the pump 16 to the container 14 and into the drain tube 20. In some embodiments, the collection tube 18 may be the same collection tube 18 that was coupled to the fluid collection assembly 12. In other embodiments, an additional tube 18 may extend from a separate chamber and/or container outside of the fluid collection system 10 to the pump 16 and to the container 14. In such examples, the pump 16 may be draw a suction from a container that includes water and/or a cleaning detergent and discharge the water and/or detergent into the container 14. The water may clean or rinse the container 14, the collection tube 18, and then may be discharged from the container 14 via the drain tube 20. In some embodiments, the water and/or detergent may be drained from the container 14 and out of the fluid collection system 10 directly. In some embodiments, the cleaning detergent, the container 14, and the pump 16 are positioned within a common housing of the fluid collection system 10.

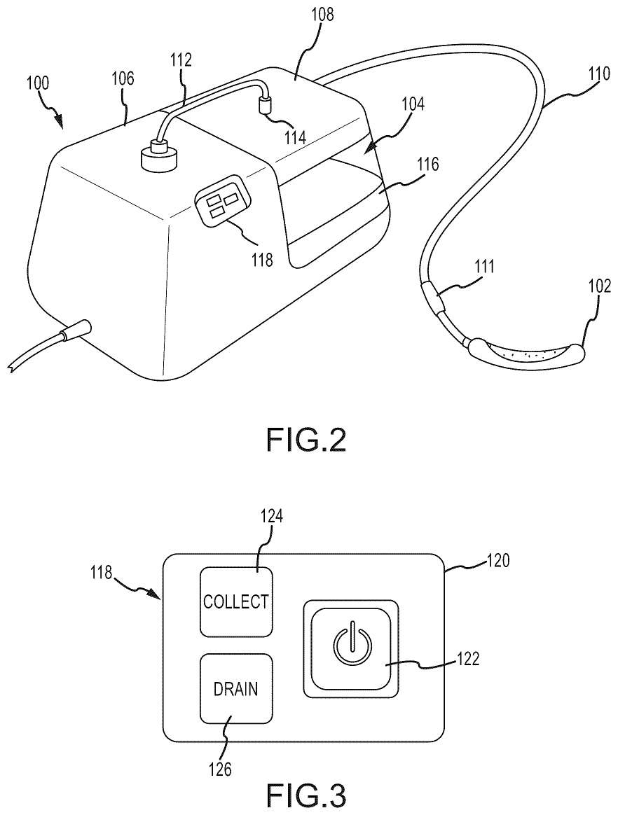

FIG. 2 is an isometric view of a fluid collection system 100, according to an embodiment. The fluid collection system 100 may include a fluid collection assembly 102 configured to receive urine from a user. The fluid collection assembly 102 may be removably attached to a container 104 of the fluid collection system 100. The container 104 may be integrated into a housing 106 that may also include a pump therein. In some embodiments, the housing 106 may be opened with a lid 108 near a top of the container 104. In some embodiments, the lid 108 may be configured to close and/or seal the opening at the top of the container 104. The fluid collection system may further include a collection tube 110 and a suction tube 112. The collection tube 110 may be disposed between the fluid collection assembly 102 and the container 104. The collection tube 110 may fluidly connect the internal volume of the container 104 with external devices or environments, including the fluid collection assembly 102. It will be understood that in some embodiments, the lid 108 may be excluded and the container 104 may define a substantially continuous volume with ports or apertures to connect the internal volume of the container 104 with the outside environment. The suction tube 112 may couple the pump suction and/or discharge to the container 104.

The pump disposed within the housing 106 may be in fluid communication with the internal volume of the container 104 via the suction tube 112. The suction tube 112 can be coupled to the lid 108 of the container 104 by a vacuum port 114. The vacuum port 114 may be configured to removably attach the suction tube 112 to the lid 108. The vacuum port 114 may be configured to create an air/liquid tight seal between the suction tube 112 and the housing 106. In some embodiments, the suction tube 112 may be configured to couple to either the suction or discharge of the pump, wherein the pump discharge is configured to provide positive pressure to the container 104 to expel the liquid from the container 104 effective to empty the container 104 of the liquid and the pump suction is configured to provide negative pressure to the container to draw fluid through the collection tube 110 into the container 104. The housing 106 may be configured to store or house internal electrical and mechanical components and the container 104. For example, the housing may include a pump (not shown in FIG. 2) configured to modulate pressure within the container 104.

The fluid collection assembly 102 may be positioned at least proximate to a urethral opening. The fluid collection assemblies shown in FIG. 2 is an example of a female fluid collection assemblies configured to collect fluid(s) from females (e.g., collect urine from a female urethra). Further examples of female fluid collection assemblies are disclosed in U.S. Pat. No. 10,390,989 issued on Aug. 27, 2019, the disclosure of which is incorporated herein, in its entirety, by this reference. However, the fluid collection assemblies, systems, and methods disclosed herein may include male fluid collection assemblies and/or devices shaped, sized, and otherwise configured to collect fluid(s) from males (e.g., collect urine from a male urethra). Examples of male fluid collection assemblies are disclosed in U.S. Provisional Patent Applications No. 63/067,542 filed on Aug. 19, 2020 and U.S. patent application Ser. No. 16/433,773 filed on Jun. 6, 2019, the disclosure of which are incorporated herein, in its entirety, by this reference.

The fluid collection assembly 102 may be disconnected from the fluid collection system 100. In some embodiments, the collection tube 110 may include a connector 111. The fluid collection assembly 102 may be disposed and replaced with a new fluid collection assembly 102.

As described in more detail below, in some embodiments, the pump is a reversible pump, configured to selectively create a vacuum in the container 104 or generate positive pressure within the container 104. For example, the pump can push air into the container 104 to expel the fluid out of the container 104 via a drain tube. When the pump is configured to generate positive pressure within the container 104, the collection tube 110 may be closed to prevent air and/or fluid from discharging through the collection tube 110. The collection tube 110 may be closed via any suitable means. For example, the collection tube 110 may include a one-way valve disposed therein. In some embodiments, the collection tube 110 may include a valve that may be manually or automatically operated to shut while the pump generates positive pressure within the container. The collection tube 110 may be pinched or otherwise prevented from discharging fluid and/or air.

In some embodiments, the collection tube 110, the suction tube 112 and/or other tubing of the fluid collection system 100 may include a flexible material such as materials tubing (e.g., medical tubing). Such material tubing may include a thermoplastic elastomer, polyvinyl chloride, ethylene vinyl acetate, polytetrafluoroethylene, flexible metal, ceramic and composite material tubing etc. The collection tube 110 may include silicon or latex. In some embodiments, the collection tube 110 may be constructed of any suitable material to be impermeable to fluids such that fluids may be drawn from the fluid collection assembly 102 and into the collection tube 110. In some embodiments, the collection tube 110 may include one or more portions that are resilient, such as by having one or more of a diameter or wall thickness that allows the collection tube 110 to be flexible.

The container 104 may be integrated into the housing 106. In some embodiments, the container 104 may include at least an inlet coupled to the collection tube 110, an outlet coupled to a drain tube and a vacuum port coupled to the pump. In some embodiments, the container 104 may be opaque or clear according to different embodiments and may include a generally rectangular front or rear profile as shown in FIG. 2 or may be cylindrical. In some embodiments, the container 104 may include a tinted color such as blue or red. The tinted window of the container may make the viewing of the urine therein less unpleasant. The container 104 may be reusable and may include a generally rigid material such as polycarbonate, plastic, rubber, metal, glass, combinations thereof, or any other suitable materials. The fluid collection container 104 may be sized and dimensioned to fit within the housing 106 and, in some embodiments, may be removable and replaceable. The container 104 may include a translucent viewing window 116. The viewing window 116 may include markings to indicate a fluid level within the fluid collection container 110.

In some embodiments, the container 104 may include a light therein. The light may be included to allow the user and/or a caretaker to check the level of fluid within the container 104 when in a dark environment. In some embodiments, the light may be an LED, LCD, incandescent, or other suitable bulb located within or proximate to the container 104. In some embodiments, the light may be located on the lid 108.

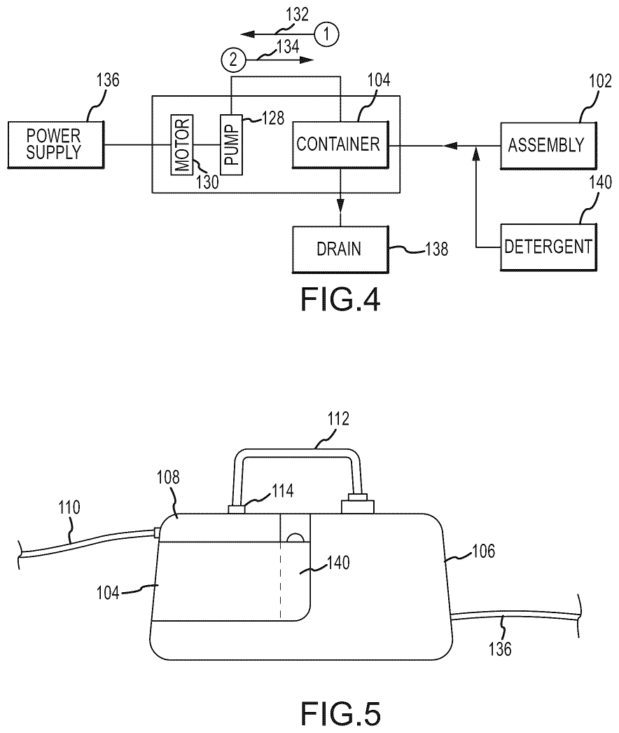

The fluid collection system may include a controller 118 configured to start the pump and switch the mode of the pump between a first mode and a second mode. Referring now to FIG. 3, the controller 118 may include a control panel 120 mounted on the housing 106. As described in greater detail with reference to FIG. 4, the fluid collection system 100 may include a one or more pumps or motors (not shown in FIGS. 2-3) to modulate the pressure within the container 104. The controller 118 may include one or more buttons for the operation of the fluid collection system 100. According to an embodiment, the controller 118 may include a power button 122 that starts and/stops the pump. In other words, the power button 122 may energize and de-energize the fluid collection system 100. The controller 118 may also include a collect button 124 that causes the pump to draw urine from the urine collection assembly 102 into the container 104. The controller 118 may also include a drain button 126 to provide high pressure air and/or fluid to the container 104 sufficient to expel the contents of the container out of a drain tube or other outlet. In some embodiments, the collect button 124 may be used to draw a cleaning detergent into the container 104 either from the collection tube 110 or a separate chamber and to provide a cleaning liquid, such as water or a cleaning solution, to flush out and clean the container 104. In some embodiments, the controller 118 and/or the control panel 120 may include a control switch or button for the light within the container 104.

Referring now to FIG. 4, the fluid collection system 100 may include one or more pumps 128 or motors 130 configured to modulate the pressure within the container 104. The pump 128 may be configured to be a bi-directional pump including a first mode 132 (as indicated by a first arrow in FIG. 4) and a second mode (as indicated by a second arrow in FIG. 4). The first mode may be configured to draw urine from the fluid collection assembly 102 into the container 104 and the second mode is configured to discharge the urine from the container 104 and clean the system. In some embodiments, the pump 128 may include multiple flow rates that allow the user to select a desired or preferred flow rate. For example, a higher flow rate can be used to obtain a more thorough flushing of the container 104 with the cleaning detergent.

In some embodiments, the pump 128 and/or motor 130 may be powered by a power supply 136. In some embodiments, the power supply 136 may include one or more of a power cord (e.g., connected to a power socket) and/or one or more batteries. In some embodiments, the power supply 136 may include a lithium ion battery. In some embodiments, the battery may be alkaline or rechargeable. In some examples, the battery may be sized and shaped to fit within the housing 106 with the pump 128 and the motor 130.

When it is desired to draw fluid from the fluid collection assembly 102, in the first mode 132 the motor 130 may cause the pump 128 to produce a negative pressure in the container 104 which draws air or fluid into the container 104 from the collection tube 110. The fluid may then collect in the container 104 until it is determined that the container should be emptied.

When it is desired to empty the contents of the container 104 and/or clean the container 104, in the second mode 134 the motor 130 may cause the pump 128 to produce a positive pressure in the container 104 which pushes air or fluid into the container 104. The motor 130 can be configured to drive a fan or impeller to draw in atmospheric air and generate high pressure air to push into the container 104. The positive pressure generated in the container 104 then forces the contents of the container 104, such as urine, through a drain 138 and into a toilet or other waste disposal unit. In some embodiments, the pump 128 suction may be coupled to a chamber 140 that includes a cleaning detergent, such as water or a cleaning solution, and the pump 128 draws the detergent into the container 104. In some embodiments, the fluid collection system 100 may cycle the detergent through the container 104 and the collection tube 110 and/or other tubing including conduit 116 for a predetermined time sufficient to clean the components and/or eliminate odors. Once the cleaning detergent is transferred from the chamber 140 to the container 104 or when the cleaning cycle is complete, the pump 128 may again provide a positive air pressure into the container 104 to force the cleaning liquid from the container 104 and into drain 138.

FIG. 5 illustrates is an isometric view of the fluid collection system 100 and the backside of the housing 106 showing chamber 140, according to some embodiments. The internal volume of the chamber 140 may be made accessible by opening the lid 108 in some embodiments. In other embodiments, the chamber 140 may be accessed through a separate opening. In some embodiments, a cleaning solution may be poured into the chamber 140. In some embodiments, the cleaning solution may be added to the chamber 140 after the initial step of providing air pressure to expel urine from the chamber 104. The cleaning solution may include a cleaning solvent, such as at least one of isopropyl alcohol, glycerin, propylene glycol, or bleach. In some embodiments, the cleaning solution may be a solid detergent that may be added directly to the container 104 or placed into the chamber 140. The detergent may include a chelating or sequestering agent, such as sodium carbonate, phosphonates, polycarboxylates, citrates, silicates, gluconic acid, zeolites, or combinations thereof. The detergent may further include surfactants, bleach, enzymes, corrosion inhibitors, fragrances, dyes, etc. The chamber may include apertures such that the chamber 140 and the container 104 are in fluid communication.

In some embodiments, the cleaning fluid may be stored in a separate container (not shown). A tube may be in fluid communication with the pump 128 and can extend into the separate container having the cleaning fluid, such that the pump 128 may draw the cleaning fluid into the tube. In some embodiments, the tube may include the collection tube 110 when the fluid collection assembly 102 has been removed.

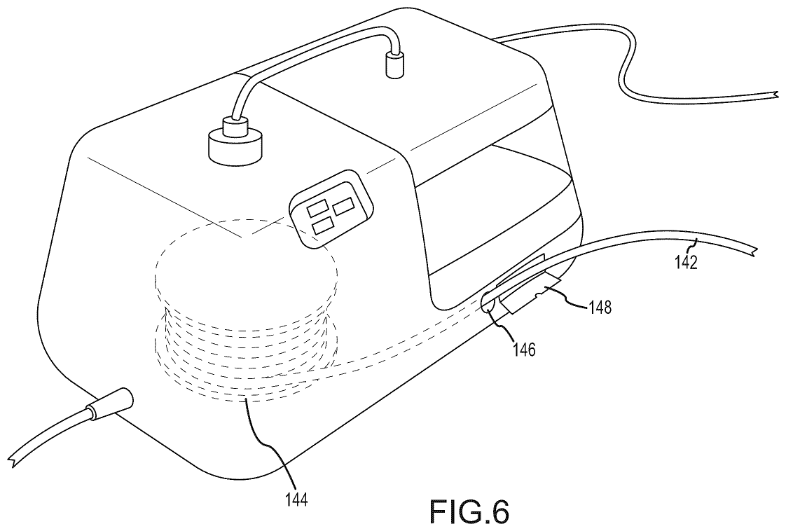

FIG. 6 illustrates is an isometric view of the fluid collection system 100 and the housing 106 with a partial cutaway view showing a drain tube 142. In some embodiments, drain 138 may include drain tube 142. In some embodiments, the housing 106 may include a compartment for storage for the drain tube 142 while it is retracted. In some embodiments, the compartment for storage includes a retracting mechanism configured to retain the drain tube 142 thereon, such that the drain tube 142 may be withdrawn to drain the container and retracts for storage.

The drain tube 142 retracting mechanism may be at least one rotatable wheel 144. When the drain tube 142 is extended outside of the housing 106, the rotatable wheel 144 may include a ratchet interlock that engages when the tension on the drain tube 142 is released such that the user does not need to maintain a tension while the drain tube 142 is extended. A first end of the drain tube 142 may be coupled to the container 104. In some embodiments, the drain tube may include a one-way valve, such that the fluid does not flow to the drain tube until the pump 128 provides sufficient positive pressure to the interior of the container 104. A second end of the drain tube 142 may be placed into a toilet or other suitable disposal container. In some embodiments, the second end of the drain tube 142 may include a valve that may be opened to drain the fluid from the drain tube 142.

After the container 104 has been drained and cleaned, the ratchet interlock may be released and the rotatable wheel 144 within the housing winds the drain tube 142 back into the housing. In some embodiments, the ratchet interlock may be released with a button 144 included on the housing. In some embodiments, the housing 106 may further include a cover 146 for the drain tube 142. The cover 146 may conceal the second end of the drain tube 142 within the housing while not in use, and ensure that the second end of the drain tube 142 remains available and doesn't retract within the housing 106.

According to an embodiment, in the second mode, the fluid collection system is configured to expel fluid in the container 104 by pushing relatively higher pressure air or fluid into the container 104. In the second mode, the motor 130 can be activated to actuate the fan 174 to push air to the conduit 116. The high pressure air travels through the conduit 116 to the container 104. The positive pressure generated in the container 104 can then push out the fluid in the container 104 via the drain 138.

In other words, a first mode of the fluid collection system 100 may also be configured to flush the container 104 with detergent. According to an embodiment, upon activation of the pump 128, a vacuum formed in the collection tube 110 draws the cleaning solution through the collection tube and into the container. Once a desired amount of cleaning solution has been transferred to the container 108, the fluid collection system 100 can again enter the second mode in order to generate positive pressure within the container 104 to expel the cleaning solution through the drain 138. In some embodiments, selection of the modes is done by a user, for example by actuating the buttons included on the control panel 120 as shown in FIG. 3. In some embodiments, the fluid collection system 100 may be configured to automatically switch modes based on, for example, a time duration or a detected fluid level within the container 104.

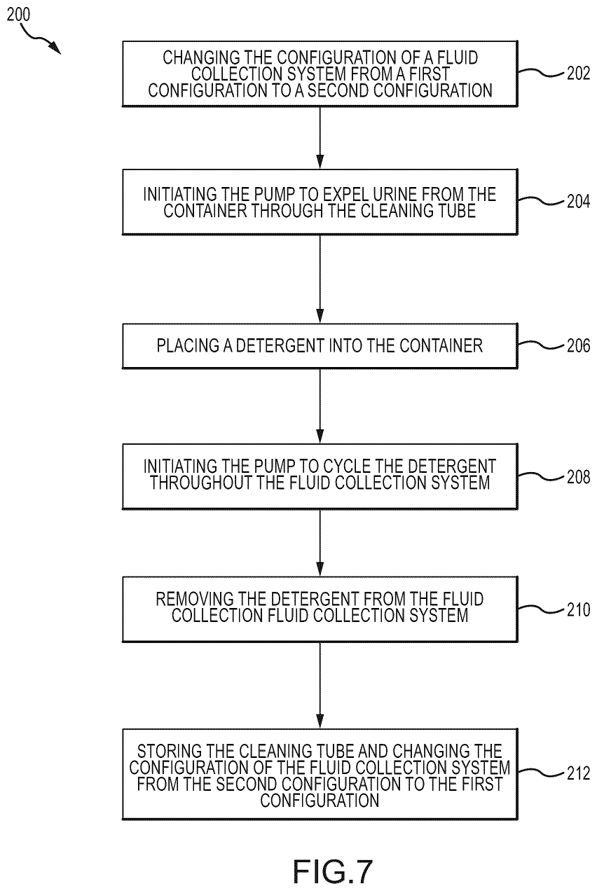

FIG. 7 is a process flow diagram of a method 200 for cleaning a fluid collection system, according to an embodiment. Some or all of the acts of method 200 may be performed using the fluid collection system 100 and/or some of the components included in the fluid collection system 100 discussed above. The method 200 may be initiated in response to a desire or need to empty and clean a container of a fluid collection system. At act 202, configuration of the fluid collection system may be changed from a first configuration to a second configuration. The fluid collection system may include a fluid collection assembly configured to receive urine from a user, a container configured to store the urine collected from the fluid collection assembly, and a collection tube disposed between the fluid collection assembly and the container. The fluid collection system may also include a cleaning tube configured to expel the urine from the container and a bi-directional pump. The pump may operate in a first mode to draw urine from the fluid collection assembly into the container and a second mode to drain the urine and clean the fluid collection system. The first mode of the pump corresponds to the first configuration of the fluid collection system and the second mode of the pump corresponds to the second configuration of the fluid collection system. Act 302 may further include removing and disposing of the fluid collection assembly.

At act 204, the pump may be initiated to expel urine from the container through the cleaning tube. With the pump in the second mode and the fluid collection system in the second configuration, a positive pressure is generated in the container to push the urine out of the cleaning tube. At act 206, a detergent may be placed into the container. In some embodiments, the detergent may be a liquid. In other embodiments, a detergent may be a powder or other solid state. In some embodiments, placing a detergent into the container includes adding water and/or sanitizer to the container. At act 208, the pump may be initiated to cycle the detergent throughout the fluid collection system. In some embodiments, a vacuum may be drawn in the container, causing the detergent to be drawn into the container. In other embodiments, a pump may cause a positive pressure in the container to push water and/or a cleaning solution throughout the fluid collection system.

At act 210, the detergent may be removed from the fluid collection system. In some examples, modulated pressure created by the pump at act 208 causes the urine within the container to be expelled from the container into a waste receptacle (e.g., a toilet) via the cleaning tube. In some embodiments, the pump draws a cleaning fluid such as water and/or detergent into the container which ultimately flushes the urine out of the container, as opposed to being pushed out by high pressure air. At act 212, the method for cleaning a fluid collection system may further include storing the cleaning tube, and changing the configuration of the fluid collection system from the second configuration to the first configuration. The method may further include attaching a fluid collection assembly to the collection tube. At this stage, the container has been emptied and cleaned and the assembly can be returned to its original assembly.

Acts 202, 204, 206, 208, 210, and 212 of the method 200 are for illustrative purposes. For example, the acts may be performed in different orders, split into multiple acts, modified, supplemented, or combined. In an embodiment, one or more of the acts may be omitted from the method 200. While the specific embodiments discussed herein have been focused on the cleaning or configuration changes of a fluid collection system, it will be understood that the methods, systems, and assemblies discussed herein could be applied to other devices.

While various aspects and embodiments have been disclosed herein, other aspects and embodiments are contemplated. The various aspects and embodiment disclosed herein are for purposes of illustration and are not intended to be limiting.

What is claimed is:

1. A fluid collection system, comprising:
a fluid collection assembly configured to receive urine from a user;
a container configured to store the urine collected from the fluid collection assembly;
a collection tube disposed between the fluid collection assembly and the container;
a bi-directional pump, wherein the pump is configured to operate in a first mode to draw urine from the fluid collection assembly into the container and a second mode to drain the urine and introduce detergent to clean the fluid collection system; and
a chamber in fluid communication with the container, the chamber configured to hold the detergent.

2. The fluid collection system of claim 1, further comprising a drain tube coupled to the container and configured to expel the urine from the container.

3. The fluid collection system of claim 1, wherein the detergent includes a cleaning solution.

4. The fluid collection system of claim 2, wherein the container includes an inlet coupled to the collection tube, an outlet coupled to the drain tube and a vacuum port coupled to the pump.

5. The fluid collection system of claim 4, wherein the vacuum port is configured to couple to either a suction or a discharge of the pump, wherein the discharge is configured to provide positive pressure to the container to expel the liquid from the container effective to empty the container of the liquid and the suction is configured to provide negative pressure to the container to draw urine from the fluid collection assembly into the container.

6. The fluid collection system of claim 1, wherein the fluid collection system includes a control panel configured to select the mode of the pump.

7. The fluid collection system of claim 1, further comprising a compartment for storage of the drain tube, wherein the compartment includes a retracting mechanism configured to retain the drain tube thereon, such that the drain tube is withdrawn to drain the container and retracts for storage.

8. The fluid collection system of claim 1, wherein the container and the pump are positioned in a common housing.

9. The fluid collection system of claim 1, wherein the container includes a translucent viewing window.

10. The fluid collection system of claim 1, wherein the container includes a light.

11. A system for collecting and disposing urine, the system comprising:
a urine collection device configured to collect urine discharged from a user;
a container fluidly coupled to the urine collection device, the container configured to store urine, wherein the container includes a fluid inlet, a fluid outlet, and a vacuum port; and
a bi-directional pump including a first mode and a second mode, wherein the first mode is configured to draw urine from the urine collection device into the container and the second mode is configured to discharge the urine from the container and introduce detergent to clean the system.
a container for the detergent including a chamber in fluid communication with the container, the chamber configured to hold the detergent.

12. The system of claim 11, wherein while in the first mode, the pump is configured to draw the urine through the fluid inlet from the urine collection device into the container.

13. The system of claim 11, wherein while in the second mode, the pump is configured to draw the detergent into the container and expel the urine through the fluid outlet.

14. The system of claim 11, further comprising a controller configured to start the pump and switch the pump between the first mode and the second mode.

15. The system of claim 11, further comprising a drain tube coupled to the fluid outlet and configured to be in fluid communication with a waste receptacle.

16. A method for cleaning a fluid collection system, the method comprising:
changing the configuration of a fluid collection system from a first configuration to a second configuration, wherein the fluid collection system includes:
a fluid collection assembly configured to receive urine from a user;
a container configured to store the urine collected from the fluid collection assembly;
a collection tube disposed between the fluid collection assembly and the container;
a cleaning tube configured to expel the urine from the container; and
a bi-directional pump, wherein the pump is configured to operate in a first mode to draw urine from the fluid collection assembly into the container and a second mode to drain the urine and introduce a detergent to clean the fluid collection system;
initiating the pump to expel urine from the container through the cleaning tube;
placing the detergent into the container by placing the detergent into a chamber in fluid communication with the container, the chamber configured to hold the detergent;
initiating the pump to cycle the detergent throughout the fluid collection system; and
removing the detergent from the fluid collection fluid collection system.

17. The method of claim 16, wherein placing a detergent into the container includes adding sanitizer to the container.

18. The method of claim 16, wherein the detergent is a liquid detergent.

19. The method of claim 16, further comprising storing the cleaning tube and changing the configuration of the fluid collection system from the second configuration to the first configuration.
